(19) United States
(12) Patent Application Publication
(10) Pub. No.: US 2018/0035942 A1
(43) Pub. Date: Feb. 8, 2018

(54) APPARATUS FOR PROVIDING SKIN-AGING INFORMATION AND METHOD THEREOF

(71) Applicant: SAMSUNG ELECTRONICS CO., LTD., Suwon-si (KR)

(72) Inventors: June Young LEE, Seongnam-si (KR); Eui Seok SHIN, Yongin-si (KR); Seung Jun LEE, Seoul (KR)

(73) Assignee: SAMSUNG ELECTRONICS CO., LTD., Suwon-si (KR)

(21) Appl. No.: 15/388,613

(22) Filed: Dec. 22, 2016

(30) Foreign Application Priority Data
Aug. 3, 2016 (KR) 10-2016-0099075

(51) Int. Cl. A61B 5/00; G06N 99/00

(52) U.S. Cl. CPC A61B 5/443; G06N 99/005; A61B 5/0075; A61B 5/7278; A61B 5/442; A61B 2576/00

(57) ABSTRACT

An apparatus for providing skin-aging information is provided. The apparatus includes a spectrum obtainer configured to obtain a skin spectrum of a user and a processor configured to extract information of at least one of collagen content, elastin content, and keratin content from the obtained skin spectrum and generate skin-aging information of the user based on the extracted information.

### CROSS-REFERENCE TO RELATED APPLICATION

This application claims priority from Korean Patent Application No. 10-2016-0099075, filed on Aug. 3, 2016 in the Korean Intellectual Property Office, the disclosure of which is incorporated by reference herein in its entirety.

### BACKGROUND

### 1. Field

Apparatuses and methods consistent with exemplary embodiments relate to providing skin-aging information.

### 2. Description of the Related Art

As the beauty industry grows, there have been increased needs for skin care and research on skin.

Human skin ages over time in a similar manner to other organs in a human body. Such a natural aging process is referred to as intrinsic aging. In addition, the skin is directly affected by an external environment unlike other organs in the human body, and thus experiences aging associated with the environment. A major environmental factor causing skin aging is sunlight, and skin aging caused by sunlight (i.e., photo aging) is accumulated over time like intrinsic aging.

Generally, skin aging or skin elasticity is measured by measuring the degree of skin wrinkling with the naked eye, however since this requires a determination of an operator, accuracy of the measurement is reduced.

### SUMMARY

One or more exemplary embodiments provide an apparatus and a method for providing skin-aging information based on a skin spectrum.

According to an aspect of an exemplary embodiment, there is provided an apparatus for providing skin-aging information including: a spectrum obtainer configured to obtain a skin spectrum of a user; and a processor configured to extract information of at least one of collagen content, elastin content, and keratin content from the obtained skin spectrum and generate skin-aging information of the user based on the extracted information.

The skin spectrum may be a near-infrared (NIR) absorption spectrum of skin.

The processor may extract the information through a regression analysis using a pure spectrum of each of collagen, elastin, and keratin.

The processor may extract the information based on a skin spectrum-body composition relation model.

The skin spectrum-body composition relation model may be generated through machine learning based on skin spectrum training data that contains collagen content data, elastin content data, and keratin content data as target data.

The skin-aging information may include at least one of a type of skin aging, a degree of skin aging, and a degree of skin elasticity.

The processor may determine a degree of intrinsic aging of the user based on the collagen content and the elastin content.

The processor may determine the degree of intrinsic aging based on a first relation table indicating a relationship of the degree of intrinsic aging and a difference between the collagen content and the elastin content.

The processor may determine a degree of photo aging based on the keratin content.

The processor may determine the degree of photo aging based on a second relation table indicating a relationship of the keratin content and the degree of photo aging.

According to an aspect of another exemplary embodiment, there is provided a method of providing skin-aging information including: obtaining a skin spectrum of a user; extracting information of at least one of collagen content, elastin content, and keratin content from the obtained skin spectrum; and generating skin-aging information of the user based on the extracted information.

The skin spectrum may be a NIR absorption spectrum about a skin.

The extracting may include extracting the information through regression analysis using a pure spectrum of each of collagen, elastin, and keratin.

The extracting may include extracting the information based on a skin spectrum-body composition relation model.

The skin-aging information may include at least one of a type of skin aging, a degree of skin aging, and a degree of skin elasticity.

The generating the skin-aging information may include determining a degree of intrinsic aging of the user based on the collagen content the elastin content, and a first relation table indicating a relationship of the degree of intrinsic aging and a difference between the collagen content and the elastin content.

The generating the skin-aging information may include determining a degree of photo aging based on the keratin content and a second relation table indicating a relationship of the keratin content and the degree of photo aging.

According to an aspect of another exemplary embodiment, there is provided an apparatus for providing skin-aging information including: a light source configured to emit light toward skin of a user; a spectroscope configured to detect the light reflected or scattered from the skin and obtain a skin spectrum from the detected light; and a processor configured to extract information of at least one of collagen content, elastin content, and keratin content from the skin spectrum and generate skin-aging information of the user based on the extracted information.

The light emitted from the light source is near-infrared (NIR) light.

The processor may determine a degree of intrinsic aging of the user based on the collagen content and the elastin content, and determine a degree of photo aging based on the keratin content.

The apparatus may further include an interval adjuster configured to adjust an interval between a skin incident position at which the emitted light is incident on the skin and a skin scattering position at which the incident light is reflected or scattered from the skin.

The interval adjuster may adjust at least one of a position of the light source and a position of the spectroscope such that a distance that the light travels through the skin is identical to a predetermined reference value.

### BRIEF DESCRIPTION OF THE DRAWINGS

The above and/or other aspects will be more apparent by describing certain exemplary embodiments, with reference to the accompanying drawings, in which:

FIG. 1 is a block diagram illustrating an apparatus for providing skin-aging information according to an exemplary embodiment.

FIG. 2 is a block diagram illustrating an apparatus for providing skin-aging information according to another exemplary embodiment.

FIG. 3 is a block diagram illustrating an apparatus for providing skin-aging information according to another exemplary embodiment.

FIG. 4 is a block diagram illustrating an apparatus for providing skin-aging information according to another exemplary embodiment.

FIG. 5A is a diagram illustrating a method of adjusting an interval between a skin incident position and a skin scattering position according to an exemplary embodiment.

FIG. 5B is a diagram illustrating a method of adjusting an interval between a skin incident position and a skin scattering position according to another exemplary embodiment.

FIG. 6 is a flow chart showing a method of providing skin-aging information according to an exemplary embodiment.

FIG. 7 is a flow chart showing a method of providing skin-aging information according to another exemplary embodiment.

FIG. 8 is a flow chart showing a method of providing skin-aging information according to another exemplary embodiment.

FIG. 9 is a detailed flow chart showing operation S810 of FIG. 8 in which an interval between a skin incident position and a skin scattering position is adjusted.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

Expressions such as “at least one of,” when preceding a list of elements, modify the entire list of elements and do not modify the individual elements of the list.

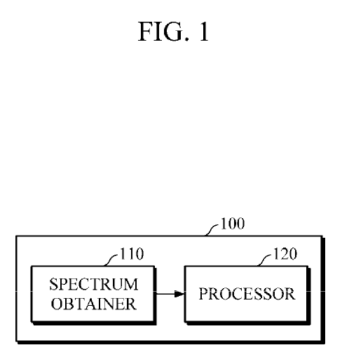

FIG. 1 is a block diagram illustrating an apparatus for providing skin-aging information 100 according to an exemplary embodiment. The apparatus 100 may generate skin-aging information of a user based on a skin spectrum of the user and provides the user with the generated skin-aging information. The apparatus 100 may be implemented with a software module, or may be manufactured in the form of a hardware chip to be mounted on an electronic device. The electronic device may include a mobile phone, a smart phone, a tablet PC, a notebook computer, a personal digital assistant (PDA), a portable multimedia player (PMP), a navigation system, an MP3 player, a digital camera, a wearable device, and the like. However, the electronic device is not limited thereto, and may include a variety of devices.

Referring to FIG. 1, the apparatus for providing skin-aging information 100 may include a spectrum obtainer 110 and a processor 120.

The spectrum obtainer 110 may obtain a skin spectrum of the user. In this case, the skin spectrum may be a near-infrared (NIR) absorption spectrum of skin that is obtained by measuring NIR emitted toward the skin of the user. However, the skin spectrum is not limited thereto, and may be a NIR transmittance spectrum of skin or NIR reflectance spectrum of a skin.

According to an exemplary embodiment, the spectrum obtainer 110 may receive skin spectrum information of the user from an external device by performing communication with the external device. For example, the spectrum obtainer 110 may receive the skin spectrum information of the user from the external device through communication such as Bluetooth communication, Bluetooth Low Energy (BLE) communication, Near Field Communication (NFC) communication, wireless local area network (WLAN) communication, Zigbee communication, Infrared Data Association (IrDA) communication, Wi-Fi Direct (WFD) communication, ultra-wideband (UWB) communication, Ant+ communication, WIFI communication, and Radio Frequency Identification (RFID) communication. However, this is only an example, and the communication is not limited thereto.

The external device may include a mobile device, a smart phone, a tablet PC, a notebook computer, a PDA, a PMP, a navigation system, an MP3 player, a digital camera, and a wearble device. However, the external device is not limited thereto, and may include various devices which may store skin spectrum information of the user.

The processor 120 may extract content information of body components that include collagen, elastin, and keratin from the skin spectrum.

According to an exemplary embodiment, the processor 120 may extract content information of each body component from the skin spectrum through regression analysis. For example, the processor 120 may resolve the skin spectrum into individual component spectrums and extract the content information of each body component from the resolved individual component spectrums through regression analysis using a pure spectrum of each body component.

According to another exemplary embodiment, the processor 120 may extract content information of each body component based on a skin spectrum-body composition relation model. The skin spectrum-body composition relation model may use a relationship between the skin spectrum and content of each body component which is defined in the model. The skin spectrum-body composition relation model may be constructed by machine learning in which skin spectrum data for training is an input and content data of each body component (e.g., collagen content data, elastin content data, and keratin content data) corresponding to the skin-spectrum data for training is a target.

A machine learning algorithm may be one of a Neural Network, a Decision Tree, a Genetic Algorithm, Genetic Programming, K-Nearest Neighbor, a Radial Basis Function Network, a Random Forest, a Support Vector Machine, and deep-learning.

The processor 120 may generate skin-aging information of the user based on the extracted content information of each body component. For example, the skin-aging information may include a type of skin aging (e.g., intrinsic aging or photo aging), a degree of skin aging, and a degree of skin elasticity.

Amounts of collagen, elastin, and keratin contained in the skin affect elasticity and aging of the skin. Collagen content and elastin content are associated with intrinsic aging, and keratin content is associated with photo aging. Specifically, in the case of intrinsic aging, elastin is reduced at a higher rate compared to collagen so that a value of the amount of collagen minus the amount of elastin tends to change from a positive value to a negative value with aging. In the case of photo aging, an epidermis gets thicker and the amount of keratin in the epidermis increases.

Accordingly, the processor 120 may determine the degree of intrinsic aging based on collagen content information and elastin content information, and may determine the degree of photo aging based on keratin content information. In this case, the processor 120 may use a relation table (hereinafter, referred to as a first relation table) in which a relationship of the degree of intrinsic aging and a difference between collagen content and elastin content in skin is defined and a relation table (hereinafter, a second relation table) in which a relationship of keratin content in skin and photo aging is defined. The first and second relation tables may be experimentally derived.

In addition, the processor 120 may calculate a combined degree of aging combining intrinsic aging and photo aging based on the degree of intrinsic aging and the degree of photo aging, and may determine a degree of skin elasticity based on the calculated combined degree of aging. In this case, the processor 120 may use a relation table (hereinafter, a third relation table) in which a relationship of a combined degree of aging and a degree of skin elasticity is defined. The third relation table may be experimentally derived.

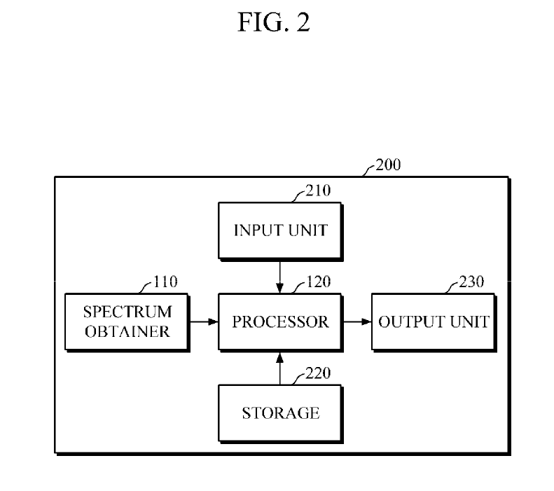

FIG. 2 is a block diagram illustrating an apparatus for providing skin-aging information according to another exemplary embodiment.

Referring to FIG. 2, an apparatus for providing skin-aging information 200 may selectively include an input unit (e.g., input interface) 210, a storage 220, and an output unit (output interface) 230 in addition to the components of the apparatus for providing skin-aging information 100 shown in FIG. 1.

The input unit 210 may receive various manipulation signals from the user. According to an exemplary embodiment, the input unit 210 may include a key pad, a dome switch, a touch pad (resistive type/capacitive type), a jog wheel, a jog switch, and a hardware button. In particular, a touch pad having a mutually layered structure with a display may be referred to as a touch screen.

The storage 220 may store programs or instructions for operating the apparatus for providing skin-aging information 200, and may store input/output data. In addition, the storage 220 may store the skin spectrum-body composition relation model and the first to third relation tables, which are previously constructed. In this case, as described above with reference to FIG. 1, the skin spectrum-body composition relation model may be constructed by machine learning in which skin spectrum data for training is input and content data of each body component (for example, collagen content data, elastin content data, and keratin content data) corresponding to the skin-spectrum data for training is a target. The first to third relation tables may be experimentally derived.

The storage 220 may include a flash memory type memory, a hard disk type memory, a multimedia card micro type memory, a card type memory (for example, secure digital (SD) or extreme digital (XD) memory), random access memory (RAM), static RAM (SRAM), read-only memory (ROM), electrically erasable programmable ROM (EEPROM), programmable ROM (PROM), a magnetic memory, a magnetic disk, and an optical disk. In addition, the apparatus for providing skin-aging information 200 may operate an external storage medium, such as a web storage, which serves as the storage 220 on the Internet.

The output unit 230 may output skin-aging information of the user. According to an exemplary embodiment, the output unit 230 may output the skin-aging information in at least one of an audible manner, a visual manner, and a tactile manner. For example, the output unit 230 may output an estimated result of weight by using voice, a text, and a vibration. To this end, the output unit 230 may include a display, a speaker, and a vibrator.

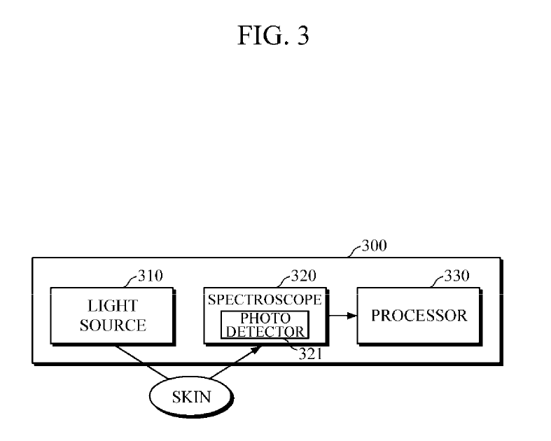

FIG. 3 is a block diagram illustrating an apparatus for providing skin-aging information according to another exemplary embodiment.

Referring to FIG. 3, an apparatus for providing skin-aging information 300 may include a light source 310, a spectroscope 320, and a processor 330. The spectrum obtainer 110 illustrated in FIGS. 1 and 2 may be implemented by the spectroscope 320, or the combination of the light source 310 and the spectroscope 320.

The light source 310 may emit light toward the skin of a user. The light emitted from the light source 110 may be NIR in a band of 1500 nm to 1900 nm or 2000 nm to 2400 nm. According to an exemplary embodiment, the light source 110 may include a light emitting diode (LED) or a laser diode.

The spectroscope 320 may measure a skin spectrum by detecting scattered light that is reflected from the skin of the user. To this end, the spectroscope 320 may include a photo detector 321. According to an exemplary embodiment, the photo detector 321 may include a photo diode, a photo transistor (PTr), or a charge-couple device (CCD) to detect the scattered light reflected from the skin of the user.

The skin spectrum measured by the spectroscope 320 may be a skin absorption spectrum. However, the skin spectrum is not limited thereto, and may be a skin transmittance spectrum or a skin reflectance spectrum.

In FIG. 3, the light source 310 and the spectroscope 320 are illustrated as separate elements. However, the present exemplary embodiment is not limited thereto, and the light source 310 may be integrated into the spectroscope 320.

The processor 330 may extract content information of body components including collagen, elastin, and keratin from the skin spectrum.

According to an exemplary embodiment, the processor 330 may extract content information of each body component from the skin spectrum through regression analysis. For example, the processor 330 may resolve the skin spectrum into individual component spectrums and extract content information of each body component from the resolved individual component spectrums through regression analysis using a pure spectrum of each body component. The pure spectrum may refer to a spectrum of pure material (e.g., collagen, elastin, and keratin) of unit mass that is measured by emitting NIR toward the pure material of unit mass.

According to another exemplary embodiment, the processor 330 may extract content information of each body component from the skin spectrum based on a skin spectrum-body composition relation model. As described above, the skin spectrum-body composition relation model may be constructed by machine learning in which skin spectrum data for training is an input and content data of each body component (e.g., collagen content data, elastin content data, and keratin content data) corresponding to the skin-spectrum data for training is a target.

The processor 330 may generate skin-aging information of the user based on the extracted content information of each body component. In this case, the skin-aging information may include a type of skin aging (intrinsic aging/photo aging), a degree of skin aging, and a degree of skin elasticity.

For example, the processor 330 may determine the degree of intrinsic aging by using the collagen content information, the elastin content information, and the first relation table, and may determine the degree of photo aging by using the keratin content information and the second relation table.

In addition, the processor 330 may calculate a combined degree of aging combining intrinsic aging and photo aging based on the degree of intrinsic aging and the degree of photo aging, and may determine a degree of skin elasticity based on the calculated combined degree of aging. In this case, the processor 330 may use the third relation table.

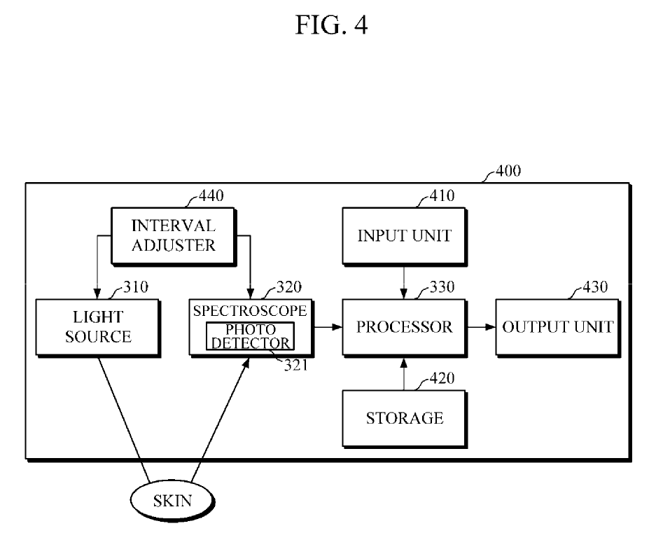

FIG. 4 is a block diagram illustrating an apparatus for providing skin-aging information according to another exemplary embodiment.

Referring to FIG. 4, an apparatus for providing skin-aging information 400 may selectively include an input unit 410, a storage 420, an output unit 430, and an interval adjuster 440 in addition to the components of the apparatus for providing a skin-aging information 300 shown in FIG. 3. Since the input unit 410, the storage 420, and the output unit 430 are identical to the input unit 210, the storage 220, and the output unit 230 described with reference to FIG. 2, details thereof will be omitted in the following description.

The interval adjuster 440 may adjust an interval between a position (hereinafter, referred to as a skin incident position) at which light irradiated from the light source 310 is incident on skin and a position (hereinafter, referred to as a skin scattering position) at which the incident light is reflected and scattered from the skin. According to an exemplary embodiment, the interval adjuster 440 may adjust the interval between the skin incident position and the skin scattering position by controlling the light source 310 and the photo detector 321 such that a path length, that is, a distance travelled by the light emitted from the light source 310 in the skin of the user, is identical to a predetermined reference value. The predetermined reference value may be set to be in a range of 0.5 mm to 3 mm such that the light emitted from the light source 310 sufficiently passes through a dermis layer of the skin. However, the predetermined reference value is not limited thereto, and may be set to be various values depending on the use and performance of a system. The path length, that is, the distance of the light that travels through the skin of the user, may be calculated by analyzing a skin spectrum.

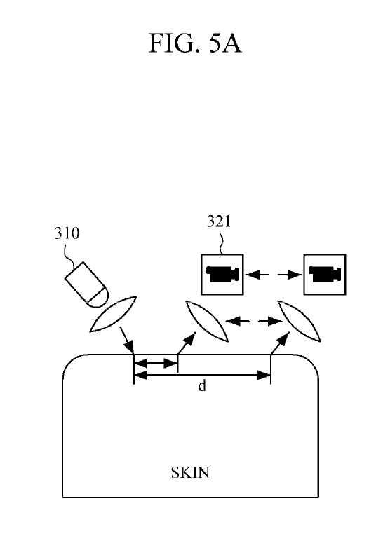

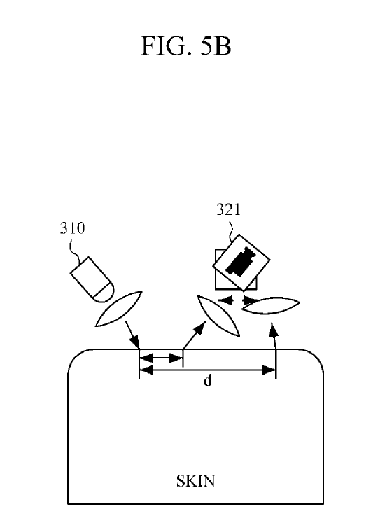

FIG. 5A is a diagram illustrating a method of adjusting an interval between a skin incident position and a skin scattering position according to an exemplary embodiment.

Referring to FIG. 5A, the photo detector 321 may be implemented in the form of a translational stage. The interval adjuster 440 may adjust an interval d between the skin incident position and the skin scattering position by moving the photo detector 321 implemented in the form of a translational stage. In this case, the light source 310 may be fixed.

FIG. 5B is a diagram illustrating a method of adjusting an interval between a skin incident position and a skin scattering position according to another exemplary embodiment.

Referring to FIG. 5B, the photo detector 321 may be implemented in the form of a rotational stage. In this case, the interval adjuster 440 may adjust the interval d between the skin incident position and the skin scattering position by rotating the photo detector 321 implemented in the form of a rotational stage. In this case, the light source 310 may be fixed.

Although the light source 310 is fixed and the photo detector 321 is implemented as a translational stage or a rotational stage as illustrated in FIGS. 5A and 5B, the exemplary embodiment is not limited thereto. For example, the photo detector 321 may be fixed, and the light source 310 may be provided as a translational stage or a rotational stage to move or rotate. Alternatively, each of the light source 310 and the photo detector 321 may be provided as a translational stage or a rotational stage so that the light source 310 and the photo detector 321 may individually move or rotate.

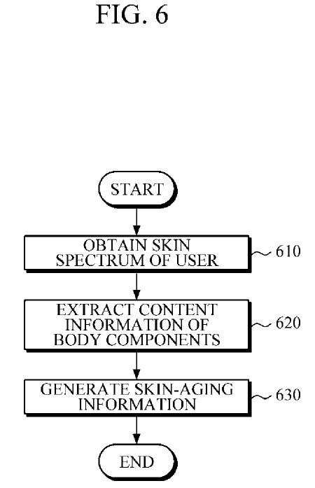

FIG. 6 is a flow chart showing a method of providing skin-aging information according to an exemplary embodiment.

Referring to FIGS. 1 to 6, the apparatus for providing skin-aging information 100 may obtain a skin spectrum of a user (operation S610). In this case, the skin spectrum may be a NIR absorption spectrum of skin that is obtained by measuring NIR emitted toward the skin of the user. However, the skin spectrum is not limited thereto, and may be a NIR transmittance spectrum of the skin or NIR reflectance spectrum of the skin.

According to an exemplary embodiment, the apparatus for providing skin-aging information 100 may receive skin spectrum information of the user from an external device by performing communication with the external device. For example, the apparatus 100 may receive the skin spectrum information of the user from the external device through communication, such as Bluetooth communication, BLE communication, NFC communication, WLAN communication, Zigbee communication, IrDA communication, WFD communication, UWB communication, Ant+ communication, WIFI communication, and RFID communication. However, this is only an example and the communication is not limited thereto.

The apparatus 100 may extract content information of body components including collagen, elastin, and keratin from the skin spectrum (operation S620).

According to an exemplary embodiment, the apparatus for providing skin-aging information 100 may extract content information of each body component from the skin spectrum through regression analysis. For example, the apparatus 100 may resolve the skin spectrum into individual component spectrums and extract the content information of each body component from the resolved individual component spectrums through regression analysis using a pure spectrum of each body component.

According to another exemplary embodiment, the apparatus for providing skin-aging information 100 may extract the content information of each body component based on a skin spectrum-body composition relation model. The skin spectrum-body composition relation model may use a relationship between the skin spectrum and the content of each body component which is defined in the model. The skin spectrum-body composition relation model may be constructed by machine learning in which skin spectrum data for training is an input and the content data of each body component (for example, collagen content data, elastin content data, and keratin content data) corresponding to the skin-spectrum data for training is a target.

The apparatus for providing skin-aging information 100 may generate skin-aging information of the user based on the extracted content information of each body component (operation S630). For example, the skin-aging information may include a type of skin aging (intrinsic aging/photo aging), a degree of skin aging, and a degree of skin elasticity.

For example, the apparatus for providing skin-aging information 100 may determine the degree of intrinsic aging based on the collagen content information and the elastin content information, and may determine the degree of photo aging based on the keratin content information. In this case, the apparatus 100 may use the first relation table in which a relationship of the degree of intrinsic aging and a difference between the collagen content and the elastin content in skin is defined and the second relation table in which a relationship of the keratin content in skin and the degree of photo aging is defined.

The apparatus for providing skin-aging information 100 may calculate a combined degree of aging combining intrinsic aging and photo aging based on the degree of intrinsic aging and the degree of photo aging, and may determine the degree of skin elasticity based on the calculated combined degree of aging. In this case, the apparatus 100 may use the third relation table.

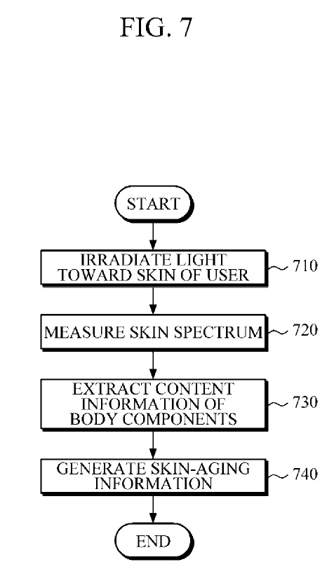

FIG. 7 is a flow chart showing a method of providing skin-aging information according to another exemplary embodiment.

Referring to FIGS. 3 and 7, the apparatus for providing skin-aging information 300 may emit light toward skin of a user (operation S710). The light emitted from the apparatus 300 may be NIR in a band of 1500 nm to 1900 nm or 2000 nm to 2400 nm.

The apparatus for providing skin-aging information 300 may measure a skin spectrum by detecting light that is scattered or reflected from the skin of the user (operation S720). Meanwhile, the skin spectrum measured by the apparatus 300 may be a skin absorption spectrum, but the skin spectrum is not limited thereto, and may be a skin transmittance spectrum or a skin reflectance spectrum.

The apparatus for providing skin-aging information 300 may extract content information of body components including collagen, elastin, and keratin from the skin spectrum (operation S730).

According to an exemplary embodiment, the apparatus for providing skin-aging information 300 may extract content information of each body component from the skin spectrum through regression analysis. For example, the apparatus 300 may resolve the skin spectrum into individual component spectrums and extract the content information of each body component from the resolved individual component spectrums through regression analysis using a pure spectrum of each body component.

According to another exemplary embodiment, the apparatus for providing skin-aging information 300 may extract the content information of each body component based on a skin spectrum-body composition relation model.

The apparatus for providing skin-aging information 300 may generate skin-aging information of the user based on the extracted content information of each body component (operation S740). In this case, the skin-aging information may include a type of skin aging (intrinsic aging/photo aging), a degree of skin aging, and a degree of skin elasticity.

For example, the apparatus for providing skin-aging information 300 may determine the degree of intrinsic aging based on collagen content information, elastin content information, and the first relation table, and may determine the degree of photo aging based on keratin content information and the second relation table.

In addition, the apparatus for providing skin-aging information 300 may calculate a combined degree of aging combining intrinsic aging and photo aging based on the degree of intrinsic aging and the degree of photo aging, and may determine the degree of skin elasticity based on the calculated combined degree of aging. In this case, the apparatus 300 may use the third relation table.

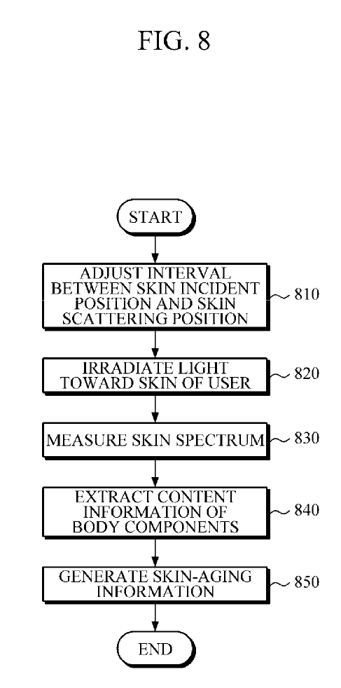

FIG. 8 is a flow chart showing a method of providing skin-aging information according to another exemplary embodiment.

Referring to FIGS. 4 and 8, the apparatus for providing skin-aging information 400 may adjust an interval between a skin incident position at which a light emitted from the light source 310 is incident on skin and a skin scattering position at which the incident light is reflected and scattered from the skin (operation S810). According to an exemplary embodiment, the apparatus 400 may adjust the interval between the skin incident position and the skin scattering position by controlling the light source 310 and the photo detector 321 such that a path length, that is, a distance that the light travels in the skin of the user, is identical to a predetermined reference value. The predetermined reference value may be set to be in a range of 0.5 mm to 3 mm such that the light irradiated from the light source 310 sufficiently passes through a dermis layer of the skin. However, the predetermined reference value is not limited thereto, and may be set to be various values depending on the use and performance of a system.

The apparatus for providing skin-aging information 400 may emit light toward the skin of a user (operation S820). The light emitted from the apparatus 400 may be NIR in a band of 1500 nm to 1900 nm or 2000 nm to 2400 nm.

The apparatus for providing skin-aging information 400 may measure a skin spectrum by detecting light that is scattered or reflected from the skin of the user (operation S830).

The apparatus for providing skin-aging information 400 may extract content information of body components including collagen, elastin, and keratin from the skin spectrum (operation S840).

According to an exemplary embodiment, the apparatus for providing skin-aging information 400 may extract content information of each body component from the skin spectrum through regression analysis. For example, the apparatus 400 may resolve the skin spectrum into individual component spectrums and extract the content information of each body component from the resolved individual component spectrums through regression analysis using a pure spectrum of each body component.

According to another exemplary embodiment, the apparatus for providing skin-aging information 400 may extract the content information of each body component based on a skin spectrum-body composition relation model.

The apparatus for providing skin-aging information 400 may generate skin-aging information of the user based on the extracted content information of each body component (operation S850). In this case, the skin-aging information may include a type of skin aging (e.g., intrinsic aging or photo aging), a degree of skin aging, and a degree of skin elasticity.

For example, the apparatus for providing skin-aging information 400 may determine the degree of intrinsic aging based on collagen content information, elastin content information, and the first relation table, and may determine the degree of photo aging based on keratin content information and the second relation table.

In addition, the apparatus for providing skin-aging information 400 may calculate a combined degree of aging combining intrinsic aging and photo aging based on the degree of intrinsic aging and the degree of photo aging, and may determine the degree of skin elasticity based on the calculated combined degree of aging. In this case, the apparatus 400 may use the third relation table.

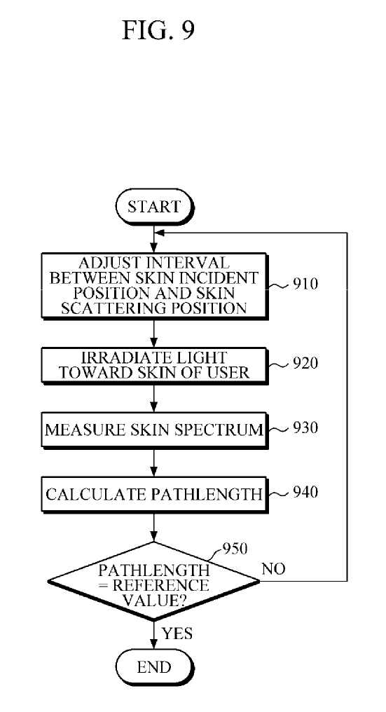

FIG. 9 is a detailed flow chart showing an operation (operation S810) in which an interval between a skin incident position and a skin scattering position is adjusted shown in FIG. 8.

Referring to FIGS. 4 and 9, the apparatus for providing skin-aging information 400 may adjust the interval between a skin incident position at which light irradiated from the light source 310 is incident on skin and a skin scattering position at which the incident light is reflected and scattered from the skin by controlling the light source 310 and the photo detector 321 (operation S910).

The apparatus for providing skin-aging information 400 may emit light toward the skin of a user according to the adjusted interval (operation S920) and measure a skin spectrum by detecting light that is reflected and scattered from the skin of the user (operation S930).

The apparatus for providing skin-aging information 400 may calculate a path length, that is, a distance that the light travels inside the skin of the user, by analyzing the measured skin spectrum (operation S940).

The apparatus for providing skin-aging information 400 may compare the calculated path length with a predetermined reference value (operation S950). The predetermined reference value may be set to be in a range of 0.5 mm to 3 mm such that the light emitted from the light source 310 sufficiently passes through a dermis layer of the skin. However, the predetermined reference value is not limited thereto, and may be set to be various values depending on the use and performance of a system.

The apparatus for providing skin-aging information 400 may terminate the operation of adjusting the interval between the skin incident position and the skin scattering position when the calculated path length is identical to the predetermined reference value, and otherwise may return to operation S910.

While not restricted thereto, an exemplary embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units of the above-described apparatuses and devices can include circuitry, a processor, a microprocessor, etc., and may execute a computer program stored in a computer-readable medium. The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for providing skin-aging information, the apparatus comprising:
a spectrum obtainer configured to obtain a skin spectrum of a user; and
a processor configured to extract information of at least one of collagen content, elastin content, and keratin content from the obtained skin spectrum and generate skin-aging information of the user based on the extracted information.

2. The apparatus of claim 1, wherein the skin spectrum is a near-infrared absorption spectrum of skin.

3. The apparatus of claim 1, wherein the processor is further configured to extract the information through regression analysis using a pure spectrum of each of collagen, elastin, and keratin.

4. The apparatus of claim 1, wherein the processor is further configured to extract the information based on a skin spectrum-body composition relation model.

5. The apparatus of claim 4, wherein the skin spectrum-body composition relation model is generated through machine learning based on skin spectrum training data that contains collagen content data, elastin content data, and keratin content data as target data.

6. The apparatus of claim 1, wherein the skin-aging information includes at least one of a type of skin aging, a degree of skin aging, and a degree of skin elasticity.

7. The apparatus of claim 1, wherein the processor is further configured to determine a degree of intrinsic aging of the user based on the collagen content and the elastin content.

8. The apparatus of claim 7, wherein the processor is further configured to determine the degree of intrinsic aging based on a first relation table indicating a relationship of the degree of intrinsic aging and a difference between the collagen content and the elastin content.

9. The apparatus of claim 1, wherein the processor is further configured to determine a degree of photo aging based on the keratin content.

10. The apparatus of claim 9, wherein the processor is further configured to determine the degree of photo aging based on a second relation table indicating a relationship of the keratin content and the degree of photo aging.

11. A method of providing skin-aging information, the method comprising:
obtaining a skin spectrum of a user;
extracting information of at least one of collagen content, elastin content, and keratin content from the obtained skin spectrum; and
generating skin-aging information of the user based on the extracted information.

12. The method of claim 11, wherein the skin spectrum is a near-infrared absorption spectrum of skin.

13. The method of claim 11, wherein the extracting comprises extracting the information through regression analysis using a pure spectrum of each of collagen, elastin, and keratin.

14. The method of claim 11, wherein the extracting comprises extracting the information based on a skin spectrum-body composition relation model.

15. The method of claim 11, wherein the skin-aging information comprises at least one of a type of skin aging, a degree of skin aging, and a degree of skin elasticity.

16. The method of claim 11, wherein the generating the skin-aging information comprises determining a degree of intrinsic aging of the user based on the collagen content the elastin content, and a first relation table indicating a relationship of the degree of intrinsic aging and a difference between the collagen content and the elastin content.

17. The method of claim 11, wherein the generating the skin-aging information comprises determining a degree of photo aging based on the keratin content and a second relation table indicating a relationship of the keratin content and the degree of photo aging.

18. An apparatus for providing skin-aging information, the apparatus comprising:
a light source configured to emit light toward skin of a user;
a spectroscope configured to detect the light reflected or scattered from the skin and obtain a skin spectrum from the detected light; and
a processor configured to extract information of at least one of collagen content, elastin content, and keratin content from the skin spectrum and generate skin-aging information of the user based on the extracted information.

19. The apparatus of claim 18, wherein the light emitted from the light source is near-infrared light.

20. The apparatus of claim 18, wherein the processor is further configured to determine a degree of intrinsic aging of the user based on the collagen content and the elastin content, and determine a degree of photo aging of the user based on the keratin content.

21. The apparatus of claim 18, further comprising an interval adjuster configured to adjust an interval between a skin incident position at which the emitted light is incident on the skin and a skin scattering position at which the incident light is reflected or scattered from the skin.

22. The apparatus of claim 21, wherein the interval adjuster is further configured to adjust at least one of a position of the light source and a position of the spectroscope such that a distance that the light travels through the skin is identical to a predetermined reference value.
